(19) United States
(12) Patent Application Publication
(10) Pub. No.: US 2005/0054930 A1
(43) Pub. Date: Mar. 10, 2005

(54) Sonoelastography using power Doppler

(75) Inventors: Ian W. Rickets, Newport-on-Tay (GB); Stephen J. McKenna, Tayport (GB); Stuart M. Dickson, Lightwater (GB); Alfred Cuschieri, St. Andrews (GB); Tim Frank, Wormit (GB); Asif Iqbal, Dundee (GB)

Correspondence Address: DICKE, BILLIG & CZAJA, P.L.L.C., FIFTH STREET TOWERS, 100 SOUTH FIFTH STREET, SUITE 2250, MINNEAPOLIS, MN, 55402, US

(73) Assignee: THE UNIVERSITY COURT OF THE UNIVERSITY OF DUNDEE

(21) Appl. No.: 10/658,099

(22) Filed: Sep. 9, 2003

(51) Int. Cl. A61B 8/02

(52) U.S. Cl. 600/453

(57) ABSTRACT

A technique for imaging relative elastic properties combines B-scan and power Doppler signals to produce images of relative vibration amplitude. A method for imaging relative elastic properties of tissue includes vibrating an area of tissue of interest and capturing a power Doppler image of at least part of the vibrating tissue.

### BACKGROUND OF THE INVENTION

1. Field of the Invention

The present invention relates to a sonoelastography method for detecting lumps within tissue, in particular tumors.

2. Description of Related Art

The most common clinical method for detecting lumps within tissue, palpation, is highly subjective and dependent on the skill of the practitioner. The method exists because certain pathological conditions, such as malignant tumors, manifest themselves as changes in the tissue's mechanical stiffness. While X-ray imaging is well established for the detection of small, deeply located tumors, X-ray hazards and the desire for better performance have led to a continuing search for alternative techniques.

Diagnostic ultrasound is a potential alternative to X-rays, its limitation being that small pathological changes in tissue are difficult to discern on normal ultrasound B-scans. If, however, ultrasonic echo data is collected before and after a slight compression of the tissue, comparisons can be made between normal and pathological areas. This is possible when normal tissues exhibit relatively more movement than stiffer pathological regions. It has been suggested that benign and malignant tumors can be distinguished by elastography, i.e. the imaging of elasticity, due to their differing uniformity of elastic properties. This is discussed in the article by J. Ophir entitled “Scientists Use Finite Element Method in Developing New Cancer Detection Technique”, NASA Tech. Briefs, pages 86-86, August 1998, incorporated herein by reference. Other authors suggest that differences between healthy and pathological tissue are highlighted more clearly using rapidly changing strain. This time-dependent (i.e. viscous) response is analogous to the vibrational frequency response of the tissue. It is likely that a relatively narrow band of vibration frequencies exists for which the response in the tissue is optimum for distinguishing variations in viscoelastic properties.

Ultrasound imaging of elastic properties in the presence of vibration is known as sonoelastography. A small, stiff zone will appear as a defined region due to the difference between its motion and that of the surrounding tissue. Ultrasound sonoelastography imaging has been compared to conventional ultrasound imaging for the detection of prostate cancer in vitro, with promising results; see D. Rubens et al “Sonoelastography Imaging of Prostrate Cancer: In Vitro Results” Radiology, 195:379-383, 1995, incorporated herein by reference. Although elastography and sonoelastography are not yet being used in routine clinical practice, these imaging methods have the potential to give comparable spatial resolution to standard grey-scale imaging with enhanced tissue discrimination.

Several sonoelastography techniques for imaging tissue elasticity have been proposed. A review of these various techniques is provided by Gao et al “Imaging of the Elastic Properties of Tissue—A Review”, Ultrasound Med. Biol., 22:959-77, 1996, incorporated herein by reference. Taylor et al in the article “Three Dimensional Sonoelastography: Principle and Practices” Phys. Med Biol., 2000, incorporated herein by reference, classify existing methods as (i) compression elastography (strain imaging), (ii) transient elastography and (iii) vibration sonoelastography. In compression elastography, ultrasound images are compared before and after a compression is applied to the tissue in order to compute a strain map. In transient elastography, a low-frequency transient vibration is applied and the resulting tissue displacement is detected using ultrasound before echoes from tissue boundaries occur. The third class, vibration sonoelastography, images the vibration patterns resulting when low frequency vibration is applied to the tissue. Vibration propagation within a complex organ cannot be solved analytically. However, small, stiff lesions will tend to result in decreases in vibration amplitude. The extent to which they are contrasted with the surrounding tissue will depend on their size and stiffness and on the frequency of vibration. Losses at high frequencies impose an upper limit on the vibration frequencies that can be used in practice.

Vibration amplitude imaging with low-frequency vibration was first proposed in the late 1980's, see the article “Sonoelasticity: Medical Elasticity Images Derived from Ultrasound Signals in Mechanically Vibrated Targets” by Lerner et el, Proc. 16^{th} Int. Sym. Acoustical Imaging, Vol. 19, pages 317-327, New York, 1998, incorporated herein by reference. Tissue motion can be estimated by tracking the two-dimensional image motion of the speckle produced by back-scattering in high frame-rate, real-time ultrasound. Such tracking is often based on template matching methods (e.g. correlation-based motion estimation) although other optical flow algorithms may also be applicable, see “Estimating Motion in Noisy, Textured Images” by Cooper et al, British Machine Vision Conference (BMVC), pages 585-594, 1996, incorporated herein by reference. Speckle tracking can be used to produce images of strain magnitude, as described for example in the article “Strain Rate Imaging Using Two-Dimensional Speckle Tracking” by Kaluzynski et al, IEEE Transactions on Ultrasonics, Ferroelectrics and Frequency Control, 48(4):1111-11123, July 2001, incorporated herein by reference. Finite element methods have been proposed to enable reconstruction of the spatial distribution of Young's modulus, see “Evaluation of an Iterative Reconstruction Method for Quantitative Elastography” by Doyley et al, Phys. Med. Biol., 45:1521-1540, 2000, incorporated herein by reference.

An alternative to speckle tracking is the use of real-time Doppler ultrasound. Doppler techniques measure the component of motion in the direction of ultrasound wave propagation and as such detect axial motion. Doppler ultrasound machines typically use autocorrelation estimators to estimate the mean frequency and the variance of the power spectrum, see “Doppler Ultrasound: Physics, Instrumentation, and Signal Processing” by Evans et al, Wiley, 2^{nd} edition, 1999, incorporated herein by reference. In flow Doppler, which is used for imaging blood flow for example, the mean frequency is used to estimate the mean velocity. However, under sinusoidal vibration, mean velocity gives no indication of vibration amplitude since oscillation is about a rest position. In the article “Three Dimensional Sonoelastography: Principles and Practice”, Phys. Med. Biol., 2000, incorporated herein by reference, Taylor et al make use of the relationship between vibration amplitude and flow Doppler variance. They used a scanner specially modified to display the real-time estimate of the variance of the power spectrum. Under reasonable assumptions the standard deviation of the power spectrum is linearly related to vibration amplitude. They were thus able to image the vibration amplitude by measuring this variance. Various other Doppler based techniques are described in U.S. Pat. Nos. 5,086,775 and 5,099,848, both of which are incorporated herein by reference.

As an alternative to sonoelastography, several authors have reported the use of phase-contrast magnetic resonance imaging (MRI) to visualize the mechanical properties of tissues; see Bishop et al “Magnetic Resonance Imaging of Shear Wave Propagation in Excised Tissue”, Journal of Magnetic Resonance Imaging, 8:1257-1265, 1998, incorporated herein by reference, and R. Ehman “Magnetic Resonance Elastography: Palpation by Imaging”, Proc. Int. Workshop on Soft Tissue Deformation and Tissue Palpation, October 1998, incorporated herein by reference. Images of tissue subjected to static or time varying displacement are obtained, yielding information on the 3D distribution of elasticity and viscoelasticity respectively. The results from these techniques are impressive in that small inhomogeneities can be localized. However, the method may never become broadly applicable due to the very high cost of MRI and its lack of portability.

### SUMMARY OF THE INVENTION

While many imaging techniques are known, there is room for improvement. To this end, an object of the present invention is to provide a new non-invasive atraumatic and painless system for tumor diagnosis and location, and in particular for breast or prostrate tumor diagnosis and location.

According to embodiments of the present invention, there is provided a method for detecting differences in tissue elasticity, the method comprising vibrating a region of tissue and capturing a power Doppler scan or image of that region of tissue.

Power Doppler scanning is a well-known feature of many ultrasound machines. This feature has been available for many years. Conventionally, however, power Doppler scan modes have only been used for the purposes of monitoring blood flow rates, and despite the widespread availability of power Doppler modes on ultrasound machines, using these as part of a sonoelastography technique has not been done before.

Preferably, a method according to an embodiment of the invention further involves capturing an ultrasound B-scan scan or image of at least part of the same region of tissue and using the B-scan scan or image to compensate the power Doppler image.

### BRIEF DESCRIPTION OF THE DRAWINGS

Various aspects of the invention will now be described by way of example only and with reference to the accompanying drawings, of which:

FIG. 1 is block diagram of an imaging system, according to an embodiment of the invention;

FIG. 2 is a simulated plot of the average power, R, of a power Doppler signal versus vibration amplitude for a vibration frequency of 200 Hz, according to an embodiment of the invention;

FIG. 3 is a plot that is similar to that of FIG. 1 except for a vibration frequency of 40 Hz, according to an embodiment of the invention;

FIG. 4 is an exploded view of part of the plot of FIG. 3, according to an embodiment of the invention;

FIGS. 5(a)-5(b) show co-registered B-scan and power Doppler images, according to an embodiment of the invention; and

FIGS. 6(a)-6(c) show co-registered B-scan, power Doppler and sonoelastographic images, according to an embodiment of the invention.

### DETAILED DESCRIPTION OF PREFERRED EMBODIMENTS

A method in which the present invention is embodied involves vibrating the region of a patient's body that is of interest and capturing a power Doppler image. By doing this, tumors can be detected in a simple, non-invasive manner. It should be noted that power Doppler cannot be used directly to estimate the vibration amplitude because the power Doppler signal depends on the echo strength of the region being imaged as well as its vibration amplitude. However, as a tumor imaging modality, the system is valid because an image that is the product of echo strength and vibration amplitude would be more sensitive for detecting cancer than an image of echo strength alone.

In addition, the method proposed here uses standard B-scan imaging to compensate the power Doppler signal in order to image the relative vibration amplitudes in the tissue. To do this, co-registered B-scan and power Doppler images of the same tissue are captured. This can be done simultaneously or sequentially. Then the B-scan data is processed. For example, the B-scan image could be processed by replacing each pixel brightness value by the square of the value. Next, the inverse of its value could be calculated as a fraction of the whole image mean value. The corresponding pixels in the power Doppler scan would then be adjusted by multiplication by the processed B-scan values, and may be modified by a theoretical and/or empirical scale factor. A process for correcting for a non-uniform vibration field could be incorporated into the above scheme. Alternatively, the magnitude of the power Doppler signal obtained at each pixel could be reduced by a factor corresponding to or a function of the square of the B-scan image at the corresponding pixel. The square root of the resulting pixel data is determined to provide the sonoelastographic image. This gives better visualization, so that a tumor that appears hyperechoic in B-scan and not apparent on power Doppler, despite its lower elasticity, would appear in the sonoelastographic image indicating that it is stiffer than the surrounding tissue.

It should be noted that in the context of this application the terms image or scan are intended to mean either the data that is captured by the scanner or indeed the actual image that is constructed from that data and presented on a screen.

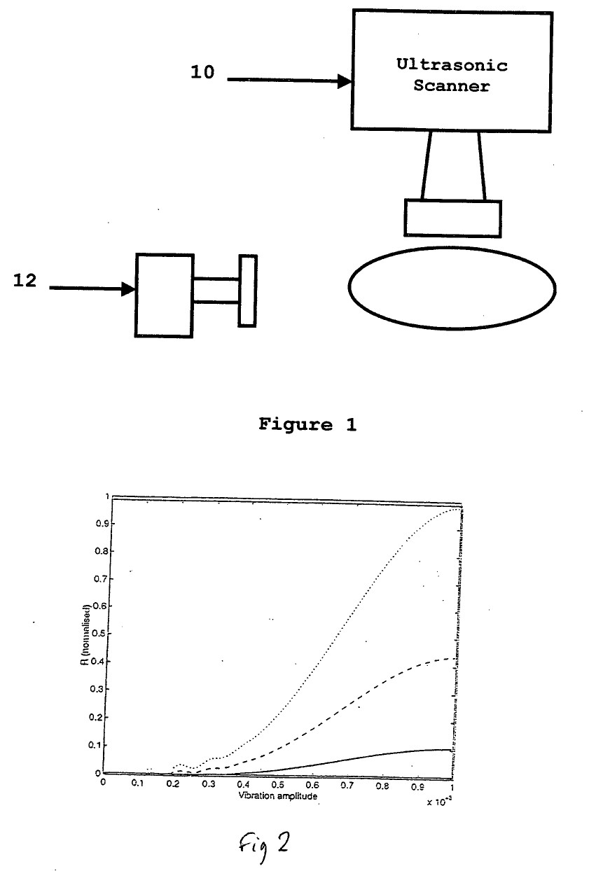

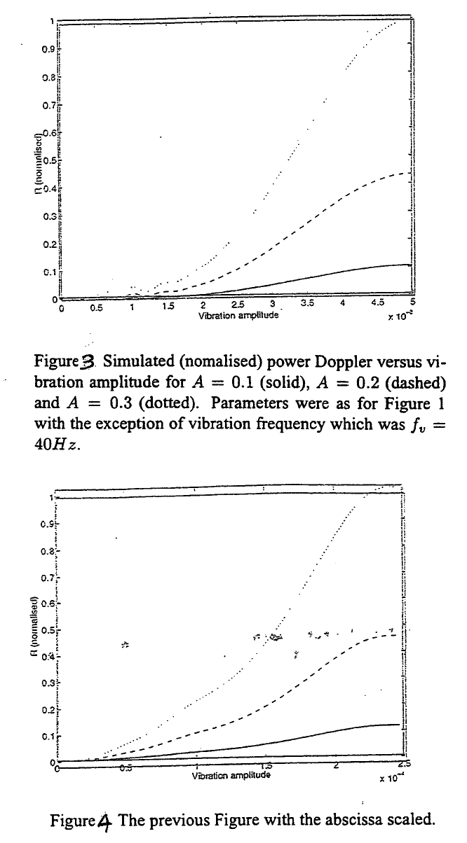

FIG. 1 shows a system for demonstrating the effectiveness of the imaging technique in which the invention is embodied. This includes an ultrasound scanner 10 for scanning the area of tissue of interest, with a vibration probe 12 for causing vibration of that area, the probe typically being adapted to be placed in direct contact with the surface that is to be vibrated. For the purposes of experiment, an Aloka SSD22O ultrasound system with a 7.5 MHz linear array probe was used as the scanner 10. This system has a power Doppler mode as standard power Doppler imaging encodes an estimate of the integrated power Doppler spectrum in pseudo-color. To vibrate the region of interest, the vibration probe 12 used was a single source using a signal generator, typically an oscillator, amplifier and an acoustic speaker. The range of vibration frequencies that can be used is limited by loss at higher frequencies. Frequencies need to be selected to enable imaging of the vibration amplitude—this will be discussed in more detail later. Typically sinusoidal vibration is used, although, as an alternative some form of polychromatic vibration, e.g. square wave vibration, could be used. This can help avoid modal patterns. The vibration systems are currently being used to conduct empirical investigation of various forms of vibration over a range of frequencies and amplitudes.

In order to assess the effectiveness of the method in which the invention is embodied, the techniques used by a standard scanner for power Doppler imaging were firstly simulated to investigate their behavior at different vibration frequencies and vibration amplitudes. Since ultrasound scanners tend to use autocorrelation estimators for color flow and power Doppler imaging, this estimation process was simulated. The simulation was similar to that used by Taylor et al to investigate the effect of vibration amplitude on estimates of the variance of the Doppler power spectrum as used for flow imaging, see “Three Dimensional Sonoelastography: Principles and Practices”, Phys. Med. Biol., 2000, incorporated herein by reference.

In pulsed Doppler ultrasound, a sequence of ultrasound pulses which together form a packet are used. The number of pulses in a packet is usually user-controlled. For the purposes of this simulation it was set to N=16. These pulses are emitted at intervals of Tₚ. Each pulse was modeled as a real wavelet (Equation (1)) where σ=173 ns and f_{c} was the center frequency:

$$p ( t ) = \frac{1}{{( 2 B {σ}^{2} )}^{1 / 2}} exp \frac{( - {t}^{2} )}{2 {σ}^{2}} cos ( 2 {Bf}_{c} t ) \qquad ( 1 )$$

The round-trip time taken from the ultrasound transducer to a scatterer at distance d₀ and back is t_{d0}=2d_{O}/c where c is the speed of sound. The mean speed of sound in tissue varies from approximately 1446 ms⁻¹ in fat to approximately 1556 ms⁻¹ in spleen, for example. The simulations here used c=1540 ms⁻¹, which is an average value used in some scanners, as discussed in “Ultrasound Imaging and Its Modelling” by Jensen, Imaging of Complex Media with Acoustic and Seismic Waves, Topics in Applied Phys, Springer Verlag, 2000, incorporated herein by reference. A scatterer under forced vibration was modeled as undergoing sinusoidal motion about a rest position at distance d₀ from the ultrasound transducer with peak vibration amplitude Eₘ and vibration frequency fᵥ Its distance d(t) from the transducer at time t is therefore given by:
d(t)=d₀−Eₘsin2Bfᵥt (2)
The backscatter, e(t), received from a single pulse at time t is:
e(t)=Ap(t−2d(t)/c) (3)
where A is the amplitude of the backscatter and models the echogenicity, the scattering coefficient and attenuation in the tissue. A first-difference FIR filter was applied to the backscattered pulse signals. This simulated a wall filter and attenuated the d.c. component.

Quadrature demodulation was simulated by sampling the N returning, filtered, backscattered pulses in a packet with the first pulse emitted at time t=0. Samples I˜were taken at times t_{I} (n)=nTₚ˜+td₀ and samples Qₙ were taken a quarter of a cycle later at times t_{Q}(n)=t_{I}(n)+1/(4f_{c}), where n=1 . . . N. In the experiments described here, Tₚ=1 ms. The average power, R, of the backscattered signal is given by the autocorrelation function at zero lag:

$$R = \frac{1 φ}{N} ( {I}_{n}^{2} + {Q}_{n}^{2} ) \qquad ( 4 )$$

It should be noted that R is proportional to A². Doubling the amplitude of the backscatter, for example, will quadruple the power Doppler signal.

FIG. 2 shows normalized power, R, plotted against vibration amplitude, Eₘ for a scatterer vibrated at a frequency of fᵥ=200 Hz at mean distance d₀=2 cm from a 7.5 MHz transducer. Three curves are plotted to correspond to scatterer strengths of A=0.1 (solid), A=0.2 (dashed) and A=0.3 (dotted). Together these curves illustrate that R increases with A². If the power signal can be compensated using an estimate of the scatterer strength these three curves become the same. The power Doppler signal increases monotonically with vibration amplitude over this range (≦1 mm) except at very low power. Therefore an appropriately compensated power Doppler signal could be used to image vibration amplitude in this situation. Note that larger vibration amplitudes would not be properly imaged. As will be appreciated, the amplitude and frequency of the vibration source should be selected to be appropriate for the ultrasound transducer used and the depth d₀ of the tissue of interest. This is necessary in order to ensure that the relationship between R and Eₘ is approximately monotonic and produces a meaningful image for the range of vibration amplitudes induced.

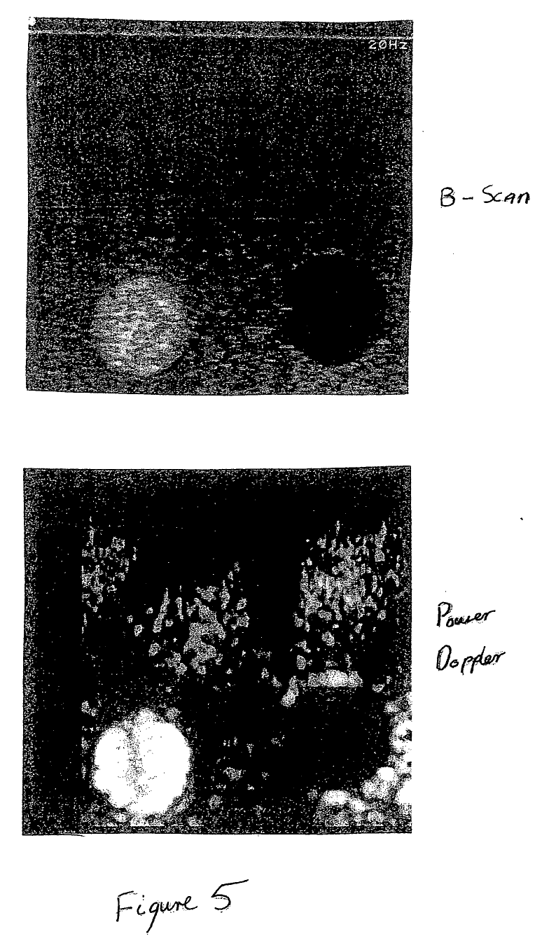

FIGS. 3 and 4 show three curves generated as in FIG. 2 but with the vibration frequency decreased to 40 Hz. The ranges of vibration amplitudes that can now be imaged reliably are different. FIG. 3 shows that the range Eₘ=0 to Eₘ=5 mm could in theory be imaged reasonably well. However, FIG. 4 shows that vibration amplitudes in the range=0 to Eₘ=250 μm could also be imaged at this depth and frequency.

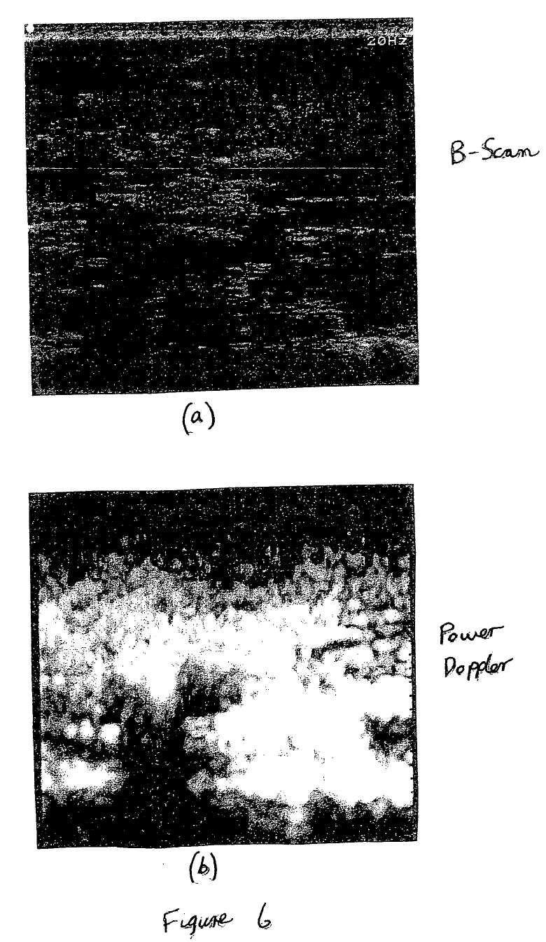

FIGS. 5(a)-5(b) show co-registered B-scan and power Doppler images of a slice through a commercially available synthetic breast phantom (supplied by Computing Imaging Reference Systems, Inc.). Vibration was applied at the base in the Figures and the transducer was at the top. The frequency of the applied vibration was 30 Hz. The images were obtained by keeping the ultrasound probe clamped in a fixed position while switching from B-scan mode to power Doppler mode. A fluid filled cyst can be seen at the lower right and a stiff lesion at the lower left. The cyst appears as a void in the B-scan and therefore also as a void in the power Doppler image. Since there is no significant backscattered signal from this region, there is no power Doppler signal. The lesion at the lower left appears contrasted in the B-scan indicating that it scatters with greater amplitude. It also appears contrasted in power Doppler signal and the extent of this contrast is due to both the increased scatter and the increased stiffness.

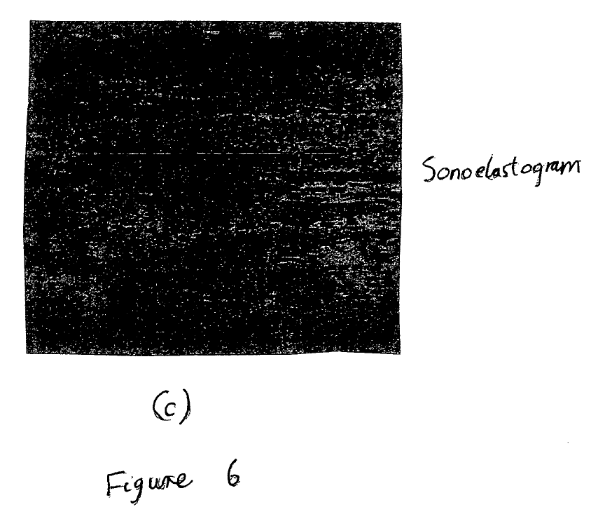

FIGS. 6(a)-6(c) show co-registered B-scan, power Doppler and sonoelastographic images of a liver phantom with a simulated tumor in the center. The sonoelastographic image was obtained by compensating the power Doppler data with squared B-scan data, and then taking the square root of each pixel to improve visualization. This can be done as long as reasonable precautions are taken to avoid electromagnetic interference. Form FIG. 6 it can be seen that the simulated tumor appears hyperechoic in the B-scan but is not apparent in the power Doppler signal as a result, despite its lower elasticity. However, it appears as a void in sonoelastographic image indicating that it is stiffer than the surrounding tissue.

Standard two-dimensional ultrasound scanners can be used to provide three-dimensional images if the 3D position and orientation of the transducer is known for each of the 2D images recorded. Free-hand 3D scanning can be used to contrast a 3D volume (voxel array) while the physician performs an examination in a normal manner. Visualization software can then provide an operator with the ability to explore the 3D volume using techniques such as any-slice imaging with transparency and 3D view-point control.

A freehand scanning system has been constructed in which the ultrasound probe's 3D position and orientation are measured using a Polhemus Fastrak electromagnetic sensor with an angular and positional accuracy of 0.5° and 0.5 mm respectively. The Polhemus device is portable and accurate. Once attached to the ultrasound probe, the Polhemus sensing system is spatially calibrated with respect to the B-scans using an object of known geometry. This spatial calibration was performed using the StradX software and a phantom constructed using a similar design to that described by Prager et al in the article “Rapid Calibration for 3-D Freehand Ultrasound”, Ultrasound in Medicine and Biology, 24(6):855-869,1998, incorporated herein by reference.

The measurements from the Polhemus sensor and the ultrasound images captured were temporally aligned using a trigger signal supplied via a footswitch. This system can be used to reconstruct 3D volumes from freehand scans and is being used to investigate the possibility of 3D sonoelastography based on simultaneously acquired B-scan and power Doppler data. High quality 3D reconstruction requires accurate calibration of the free-hand scanning device, accurate registration and data fusion. Image-based registration and fusion can improve the quality of the reconstruction and reduce speckle noise, shadowing and signal dropout, as described by Rohling et al in “Spatial Compounding of 3d Ultrasound Images”, Technical Report, CUED/F-INFENG/TR270, Univ. of Cambridge, October 1996, incorporated herein by reference, and “Automatic Registration of 3D Ultrasound Images”, Technical Report, CUED/F-INFENG/TR290, Univ. of Cambridge, May 1997, incorporated herein by reference.

Doppler imaging only measures the component of velocity in the direction of wave propagation, i.e. only axial motion can be detected. However, freehand scanning can be use to register and fuse Doppler signals from different transducer orientations. The process for forming a power Doppler is relatively slow since multi-pulse packets are used for estimation. This means that the probe can move significantly during acquisition of a single image. Probe position and orientation measurements therefore need to be interpolated over time so that columns in the 2D images obtained from the linear array probe can be aligned appropriately in 3D. The frame-rate for power Doppler imaging is often increased by reducing the area over which power Doppler signals are estimated, the remaining area being imaged in B-scan mode. During such a scan, the B-scan and power Doppler data can be recorded and registered in two separate 3D voxel arrays. Fusion of these B-scan and power Doppler volumes then compensates the power Doppler data to produce 3D sonoelastographic volume data. Each voxel is this volume could be assigned an uncertainty value based on the amount of evidence available from the 2D scans and the extent of the interpolation used. This uncertainly data could in turn provide useful information for subsequent data fusion and provide feedback to the physician performing the scan. Three-dimensional sonoelastographic imaging has the potential application of measurement of the volume distribution of tissue elastic properties. This data is required by researchers developing mathematical models of tissue and, in particular, for use in electronic tissue representation in surgical simulators.

A skilled person will appreciate that variations of the disclosed arrangements are possible without departing from the invention. For example, while the description above focuses on the location and diagnosis of tumors, and in particular breast tumors, it will be appreciated the technique in which the invention is embodied may also be used to detect other diseases that cause changes in tissue elasticity, e.g. atheromatous disease. Other applications include the measurement of tissue elasticity for use in tissue modeling studies and to provide data in virtual reality research in relation to surgical/interventional simulators. Accordingly the above description of the specific embodiment is made by way of example only and not for the purposes of limitation. It will be clear to the skilled person that minor modifications may be made without significant changes to the operation described.

1. A method for imaging relative elastic properties of tissue, the method comprising vibrating an area of tissue of interest and capturing a power Doppler image of at least part of the vibrating tissue.

2. A method as claimed in claim 1 further comprising capturing a B-scan image and using the B-scan image to compensate the power Doppler image.

3. A method as claimed in claim 2 wherein the B-scan image is captured simultaneously with the power Doppler image.

4. A method as claimed in claim 1 comprising applying a probe to the patient's body, the probe being operable to cause vibration of the area of tissue of interest.

5. A method as claimed in claim 3, wherein the probe comprises an acoustic speaker.

6. A method as claimed in claim 4 wherein the probe comprises a tissue contact pad or disc for transferring vibrations to the area of tissue of interest.

7. A method as claimed in claim 2 comprising applying a probe to the patient's body, the probe being operable to cause vibration of the area of tissue of interest.

8. A method as claimed in claim 3 comprising applying a probe to the patient's body, the probe being operable to cause vibration of the area of tissue of interest.

9. A method as claimed in claim 5 wherein the probe comprises a tissue contact pad or disc for transferring vibrations to the area of tissue of interest.
